(19) United States
(12) Patent Application Publication
(10) Pub. No.: US 2025/0352783 A1
(43) Pub. Date: Nov. 20, 2025

(54) THERAPEUTIC PULSE GENERATORS WITH CRYSTALLINE CAPACITORS

(71) Applicant: MEDTRONIC, INC., Minneapolis, MN (US)

(72) Inventors: Vladimir P. Nikolski, Blaine, MN (US); Christian S. Nielsen, River Falls, FL (US); Mark E. Viste, Brooklyn Center, MN (US)

(21) Appl. No.: 18/873,914

(22) PCT Filed: Jun. 27, 2023
(86) PCT No.: PCT/US2023/026306

§ 371 Date: Dec. 11, 2024

Related U.S. Application Data
(60) Provisional application No. 63/356,239, filed on Jun. 28, 2022.

(51) Int. Cl. A61N 1/04; A61N 1/05; A61N 1/36

(52) U.S. Cl. CPC A61N 1/0484; A61N 1/05; A61N 1/3603; A61N 1/36128

(57) ABSTRACT

Therapeutic electrical pulse delivery systems and therapeutic pulse generators are disclosed. The therapeutic electrical pulse delivery system may include a power source, a pulse generator, and a controller. The pulse generator may be operatively coupled to the power source. The pulse generator may include one or more capacitors. Each of the one or more capacitors may include a first electrode, a second electrode, and a dielectric disposed between the first electrode and the second electrode. The dielectric may include a crystalline dielectric including carbon. The controller may include one or more processors and may be operatively coupled to the power source or the pulse generator. The controller may be configured to charge the one or more capacitors of the pulse generator using the power source and cause the pulse generator to deliver a therapeutic electrical pulse using the charged one or more capacitors.

### RELATED APPLICATIONS

This application claims the benefit of U.S. Provisional Application Ser. No. 63/356,239 filed Jun. 28, 2022, which is incorporated by reference herein in its entirety.

### FIELD

The present disclosure relates to, among other things, therapeutic electrical pulse delivery systems and apparatus.

### TECHNICAL BACKGROUND

Therapeutic electrical pulse delivery systems and apparatus generally use capacitors to store energy and deliver a therapeutic pulse or shock to a patient. Capacitors may store energy in an electric field between two electrodes (e.g., a first electrode and a second electrode). Capacitors may discharge stored energy more rapidly than batteries or other power sources. Additionally, the energy stored by capacitors can have a higher voltage than energy stored and provided by typical batteries or other power sources while taking up less volume. Accordingly, capacitors may be used to provide high voltage pulses or shocks in therapeutic electrical pulse delivery systems and apparatus.

While in a lab or hospital setting the size, shape, weight, and durability of capacitors in therapeutic electrical pulse delivery systems may not be significant concern. However, for therapeutic electrical pulse delivery systems and apparatus that are designed to be implanted in or worn by a patient, concerns of size, shape, weight, and durability become prominent. Capacitors that require bulky shapes and housings may increase the size and weight of implantable and wearable devices. Furthermore, capacitors that may be subject to mechanical and/or electromechanical deformation may reduce the efficiency of therapeutic electrical pulse delivery systems and apparatus over time. Thus, capacitors and/or pulse generators that can deliver high voltage therapeutic pulses in a small low-weight form factor with high durability may be desirable.

### BRIEF SUMMARY

As described herein, therapeutic electrical pulse delivery systems and apparatus with small low-weight form factors and increased durability can be achieved using one or more capacitors that include a crystalline dielectric (e.g., diamond or silicon carbide capacitors). Capacitors that include a crystalline dielectric may reduce the mass and volume required of a capacitor to achieve a given capacitance. Additionally, crystalline dielectric capacitors may be designed in many different form factors and may be less susceptible to electromechanical deformation and degradation. Accordingly, therapeutic electrical pulse delivery systems and apparatus described herein may provide increased energy efficiency, durability, and shape variability.

Described herein, among other things, is a therapeutic electrical pulse delivery system comprising a power source; a pulse generator, and a controller. The pulse generator may be conductively coupled to the power source. The pulse generator may comprise one or more capacitors. Each of the one or more capacitors may comprise a first electrode, a second electrode and a crystalline dielectric disposed between the first electrode and the second electrode. The crystalline dielectric may comprise carbon. The controller may comprise one or more processors and may be operatively coupled to the power source or the pulse generator. The controller may be configured to charge the one or more capacitors of the pulse generator using the power source and cause the pulse generator to deliver a therapeutic electrical pulse using the charged one or more capacitors.

In general, in one aspect, the present disclosure describes a therapeutic pulse generator comprising an input, a charge bank, and an output. The input may be operatively couplable to a power source. The charge bank may be operatively coupled to the input to receive and store energy provided by the power source. The charge bank may comprise one or more capacitors. Each of the one or more capacitors may include a first electrode, a second electrode, and a crystalline dielectric disposed between the first electrode and the second electrode. The crystalline dielectric may comprise carbon. The output may be operatively coupled to the charge bank to deliver a therapeutic electrical pulse using energy stored in the charge bank.

Advantages and additional features of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF THE DRAWINGS

The following detailed description of specific embodiments of the present disclosure can be best understood when read in conjunction with the following drawings, in which:

FIG. 1 is a conceptual drawing illustrating an embodiment of a therapeutic electrical pulse delivery system in conjunction with a patient;

FIG. 2 is a conceptual drawing illustrating another embodiment of a therapeutic electrical pulse delivery system in conjunction with the patient;

FIG. 3 is schematic block diagram of the electrical components of the therapeutic electrical pulse delivery systems of FIGS. 1 and 2;

FIG. 4 is schematic block diagram of a pulse generator of the therapeutic electrical pulse delivery systems of FIGS. 1-3.

FIG. 5 is a top-down view of an embodiment of a capacitor of the pulse generator of FIG. 4;

FIG. 6 is a top-down view of another embodiment of a capacitor of the pulse generator of FIG. 4;

FIG. 7 is a top-down view of another embodiment of a capacitor of the pulse generator of FIG. 4;

FIG. 8 is a top-down view of another embodiment of a capacitor of the pulse generator of FIG. 4;

FIG. 9 is a top-down view of an embodiment of a stacked arrangement of capacitors.

The schematic drawing is not necessarily to scale.

### DETAILED DESCRIPTION

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

Generally, therapeutic electrical pulse delivery systems include electrolytic capacitors. Electrolytic capacitors are generally considered to provide a reasonable trade-off among prominent considerations in the design of therapeutic electrical pulse delivery systems (e.g., discharge energy and duration, charge time, energy efficiency, volume, mass, durability, etc.). However, electrolytic capacitors may have limitations that provide an opportunity for improvement.

One limitation may include the voltage limitations of electrolytic capacitors. Electrolytic capacitors can operate at a few hundred volts but are typically stacked in series to reach the voltage and energy requirements of therapeutic electrical pulse delivery systems. Accordingly, the high voltage requirements of therapeutic electrical pulse delivery system may result in larger devices to accommodate multiple capacitors connected in series. In contrast, capacitors that include a crystalline dielectric (e.g., crystalline capacitors) may be compatible with higher voltages and fewer capacitors may be used to achieve the same or higher voltages as electrolytic capacitors. Crystalline dielectrics can have a high dielectric strength that may allow energy to be stored in capacitors at a higher voltage with a reduced volume of dielectric compared to electrolytic capacitors. Thus, individual crystalline capacitors may also be smaller than individual electrolytic capacitors and still operate at a higher voltage than electrolytic capacitors. As a result, therapeutic electrical pulse delivery systems and/or pulse generators that include crystalline capacitors may be smaller and may allow therapeutic pulses to be delivered at higher voltages with less capacitance than those that include electrolytic capacitors. Additionally, the voltage of such therapeutic electrical pulse delivery systems can be tuned with a change in the thickness of the crystalline dielectric.

Another limitation of electrolytic capacitors may include electromechanical deformation and degradation. Electrolytic capacitors typically include an electrolyte and a housing to retain the electrolyte. Electrolytes used in electrolytic capacitors can expand and contract with use. Such expansion and contraction can cause deformations in the capacitors that that can increase charge time. In contrast, crystalline capacitors do not need or include an electrolyte. Accordingly, crystalline capacitors may not be susceptible to the same deformation and degradation as electrolytic capacitors.

As used herein, the term “crystalline dielectric” may refer to dielectric materials with a crystalline structure that include carbon. Crystalline dielectrics may include, for example, diamond, silicon carbide, or other crystal materials that include carbon. Crystalline dielectrics may also include one or more dopants such as, for example, boron, silicon, oxygen, chromium, lanthanum, phosphorus, niobium, indium, etc. As used herein, the term “crystalline capacitor” may refer to capacitors that include a crystalline dielectric. Crystalline dielectrics may reduce the amount of dielectric needed and, therefore, may reduce the mass and volume required to store a given amount of energy. Additionally, crystalline dielectrics may be less dense than dielectrics used in electrolytic capacitors such as, for example, tantalum oxide. Furthermore, because crystalline dielectrics can be deposited in planar structures, crystalline capacitors can be constructed that only include metal (e.g., electrodes) and the crystalline dielectric. Thus, the need for electrolyte may be eliminated as well as any volume needed to accommodate such electrolyte. Accordingly, crystalline capacitors may not be subject to electrolyte leakage.

Still further, because crystalline capacitors may include only metal and dielectric components, shape flexibility and variation within therapeutic electrical pulse delivery system is increased. Crystalline capacitors may include stacked layers of dielectric that may provide three-dimensional shape flexibility by changing the shape of the layers in the stack and/or the shape of the electrodes the crystalline dielectric is deposited on. For example, a crystalline capacitor could define a strip around the edge of a therapeutic electrical pulse delivery system or apparatus. Such strip crystalline capacitor may access space that may otherwise be less efficiently used. Additionally, a strip crystalline capacitor may follow a curve or wrap around a corner of a device. The strip capacitor may also be wound or rolled into a spiral shape.

According to one aspect there is provided a therapeutic electrical pulse delivery system. The therapeutic electrical pulse delivery system may include a power source, a pulse generator, and a controller. The pulse generator may be operatively coupled to the power source. The pulse generator may include one or more capacitors. Each of the one or more capacitors may include a first electrode, a second electrode, and a dielectric disposed between the first electrode and the second electrode. The dielectric may include a crystalline dielectric. The controller may include one or more processors and may be operatively coupled to the power source or the pulse generator. The controller may be configured to charge the one or more capacitors of the pulse generator using the power source and cause the pulse generator to deliver a therapeutic electrical pulse using the charged one or more capacitors.

The pulse generator may include any suitable number of capacitors. In one or more embodiments, the one or more capacitors consists of a single capacitor. The use of crystalline capacitors may allow a single capacitor to have a maximum voltage and capacitance to deliver therapeutic electrical pulses at the desired voltage. The use of a single capacitor may allow the size and cost of pulse generators and therapeutic electrical pulse delivery systems to be reduced compared to apparatus and systems that use multiple electrolytic capacitors.

Although the one or more capacitors of the pulse generator may consist of a single capacitor, arrangements that include multiple capacitors may allow for a variety of pulse generator arrangements and/or dynamic therapeutic electrical pulse delivery. The one or more capacitors may be arranged to be charged in parallel and to deliver the therapeutic electrical pulse in parallel. In other words, each of the one or more capacitors may be arranged such that a terminal of each capacitor is coupled to a first voltage node and the other terminal of each capacitor is coupled to a second voltage node. Capacitors arranged to deliver therapeutic electrical pulses in parallel may provide redundancy that allows the pulse generator to continue delivering therapeutic electrical pulses as long as one capacitor has not failed and can deliver the therapeutic electrical pulse.

In one or more embodiments, the one or more capacitors may be arranged to charge in parallel and to deliver the therapeutic electrical pulse in series. The pulse generator may include an input operatively coupled to the power source and configured to charge the one or more capacitors in parallel. The input may include one or more switches to allow power to be received from the power source to charge the one or more capacitors. The input may be controlled by the controller to charge the one or more capacitors. For example, the controller may be configured to open and close one or more switches of the input. The switches of the input may include, for example, one or more relays, transistors, digital switches, or other switch apparatus.

The pulse generator may further include an output operatively coupled to the one or more capacitors and configured to deliver the therapeutic electrical pulse from the one or more capacitors. The output may include one or more switches to allow therapeutic electrical pulses to be delivered using the one or more capacitors. The output may be controlled by the controller to deliver the therapeutic electrical pulses. For example, the controller may be configured to open and close one or more switches of the output. The switches of the output may include, for example, one or more relays, transistors, digital switches, or other switch apparatus.

In one or more embodiments, the output may be static such that the therapeutic electrical pulse is delivered from the capacitors in the same arrangement each time. In other words, the output may be configured to deliver the therapeutic electrical pulse from the capacitors in series, in parallel, or a in a combination of series and parallel. In one or more other embodiments, the output may be dynamic such that the therapeutic electrical pulse can be delivered from different combinations and arrangements of the one or more capacitors. In other words, the output may be configured to selectively deliver the therapeutic electrical pulse from the one or more capacitors in series, parallel, or in a combination of series and parallel.

For example, the output may include a plurality of switches that can be open or closed in various arrangements to deliver the therapeutic electrical pulse from the one or more capacitors. In one or more embodiments, a series of stepped-up therapeutic electrical pulses can be delivered by three capacitors. The series of stepped-up therapeutic electrical pulses may include a therapeutic electrical pulse delivered by the three capacitors in parallel. The series of stepped-up therapeutic electrical pulses may include another therapeutic electrical pulse delivered by two of the three capacitors in parallel with one another and the third of the three capacitors arranged in series with the two capacitors. The series of stepped-up therapeutic electrical pulses may include yet another therapeutic electrical pulse delivered by the three capacitors in series. Although three capacitors are used as an example, it will be appreciated that various arrangements of two capacitors or four or more capacitors can also be utilized.

The one or more capacitors may take on a variety of shapes. For example, at least one of the one or more capacitors may include one or more windings defined by the first electrode, the second electrode, and the dielectric. Further, for example, the first electrode, the second electrode, and the dielectric of at least one of the one or more capacitors may define a curved shape. Additionally, the one or more capacitors may conform to a shape of the therapeutic electrical pulse deliver system or a substrate of the therapeutic electrical pulse delivery system. In one embodiment, the therapeutic electrical pulse delivery system may include a substrate that includes a groove in at least one surface of the substrate, and the one or more capacitors may conform to a shape of the groove.

The one or more capacitors may have any suitable maximum voltage to provide therapeutic electrical pulses. Each of the one or more capacitors may have an individual maximum voltage that can be the same or different from the individual maximum voltage of other capacitors of the one or more capacitors. At least one of the one or more capacitors may have a maximum voltage of, for example, at least 100 volts, at least 300 volts, at least 1000 volts, at least 2000 volts, or any voltage therebetween. In one or more embodiments, at least one capacitor of the one or more capacitors may have a maximum voltage of at least 1000 volts.

The one or more capacitors may have any suitable maximum capacitance to provide therapeutic electrical pulses. Each of the one or more capacitors may have an individual maximum capacitance that can be the same or different from the individual maximum capacitance of other capacitors of the one or more capacitors. At least one of the one or more capacitors may have a maximum capacitance of, for example, at least 15 Farads, at least 40 Farads, at least 300 Farads, or any capacitance therebetween. In one or more embodiments, at least one capacitor of the one or more capacitors may have a maximum capacitance of at least 300 Farads.

The pulse generator may be configured to deliver the electrical pulse with any suitable voltage. The pulse generator may be configured to deliver the electrical pulse with a voltage of, for example, at least 100 volts, at least 300 volts, at least 1000 volts, at least 2000 volts, or any voltage therebetween. In one or more embodiments, the pulse generator may be configured to deliver the electrical pulse with a voltage of at least 1000 volts. The pulse generator may include a step-up converter to boost or increase the voltage received from the power source. The step-up converter may be configured to increase the voltage received from the power source to a therapeutic pulse voltage. An output voltage of the step-up converter may be adjustable based on a desired therapeutic pulse voltage. For example, the controller may be configured to adjust the output voltage of the step-up converter based on the desired therapeutic pulse voltage.

The therapeutic electrical pulse delivery system may include a housing. The pulse generator may be disposed in the housing. The housing may include a wearable device such as, a vest, a cuff, headwear, etc. In one or more embodiments, the housing may include a vest. The therapeutic electrical pulse delivery system may include any suitable medical device. In one or more embodiments, an implantable medical device includes the therapeutic electrical pulse delivery system.

According to one aspect there is provided a therapeutic pulse generator. The therapeutic pulse generator may include an input, one or more capacitors, and an output. The input may be operatively couplable to a power source. The one or more capacitors may be coupled to the input to receive and store energy provided by the power source. Each of the one or more capacitors may include a first electrode, a second electrode, and a crystalline dielectric disposed between the first electrode and the second electrode. The crystalline dielectric may include carbon. For example, the crystalline dielectric may include SiC. The output may be operatively coupled to the one or more capacitors to deliver a therapeutic electrical pulse using energy stored in the one or more capacitors.

The pulse generator may include any suitable number of capacitors. In one or more embodiments, the one or more capacitors consists of a single capacitor. The use of crystalline capacitors may allow a single capacitor to have a maximum voltage and capacitance to deliver therapeutic electrical pulses at the desired voltage. The use of a single capacitor may allow the size and cost of pulse generators to be reduced compared to apparatus and systems that use multiple electrolytic capacitors.

Although the one or more capacitors of the pulse generator may consist of a single capacitor, arrangements that include multiple capacitors may allow for a variety of pulse generator arrangements and/or dynamic therapeutic electrical pulse delivery. The one or more capacitors may be arranged to be charged in parallel and to deliver the therapeutic electrical pulse in parallel. In other words, each of the one or more capacitors may be arranged such that a terminal of each capacitor is coupled to a first voltage node and the other terminal of each capacitor is coupled to a second voltage node. In one or more embodiments, the one or more capacitors may be arranged to charge in parallel and to deliver the therapeutic electrical pulse in series.

The input may be configured to charge the one or more capacitors in parallel. The input may include one or more switches to allow power to be received from the power source to charge the one or more capacitors. The input may be controllable to charge the one or more capacitors. For example, the one or more switches may be opened or closed by a controller to control charging of the one or more capacitors. The switches of the input may include, for example, one or more relays, transistors, digital switches, or other switch apparatus.

The output may be configured to deliver the therapeutic electrical pulse from the one or more capacitors. The output may include one or more switches to allow therapeutic electrical pulses to be delivered using the one or more capacitors. The output may be controllable by a controller to deliver the therapeutic electrical pulses. For example, one or more switches of the output may be opened or closed by a controller to control the delivery of the therapeutic electrical pulse. The switches of the output may include, for example, one or more relays, transistors, digital switches, or other switch apparatus.

In one or more embodiments, the output may be static such that the therapeutic electrical pulse is delivered from the capacitors in the same arrangement each time. In other words, the output may be configured to deliver the therapeutic electrical pulse from the capacitors in series, in parallel, or a in a combination of series and parallel. In one or more other embodiments, the output may be dynamic such that the therapeutic electrical pulse can be delivered from different combinations and arrangements of the one or more capacitors. In other words, the output may be configured to selectively deliver the therapeutic electrical pulse from the one or more capacitors in series, parallel, or in a combination of series and parallel.

For example, the output may include a plurality of switches that can be open or closed in various arrangements to deliver the therapeutic electrical pulse from the one or more capacitors. In one or more embodiments, a series of stepped-up therapeutic electrical pulses can be delivered by three capacitors. The series of stepped-up therapeutic electrical pulses may include a therapeutic electrical pulse delivered by the three capacitors in parallel. The series of stepped-up therapeutic electrical pulses may include another therapeutic electrical pulse delivered by two of the three capacitors in parallel with one another and the third of the three capacitors arranged in series with the two capacitors. The series of stepped-up therapeutic electrical pulses may include yet another therapeutic electrical pulse delivered by the three capacitors in series. Although three capacitors are used as an example, it will be appreciated that various arrangements of two capacitors or four or more capacitors can also be utilized.

The one or more capacitors may take on a variety of shapes. For example, at least one of the one or more capacitors may include one or more windings defined by the first electrode, the second electrode, and the dielectric. Further, for example, the first electrode, the second electrode, and the dielectric of at least one of the one or more capacitors may define a curved shape. Additionally, the one or more capacitors may conform to a shape of the therapeutic electrical pulse deliver system or a substrate of the therapeutic electrical pulse delivery system. In one embodiment, the therapeutic electrical pulse delivery system may include a substrate that includes a groove in at least one surface of the substrate, and the one or more capacitors may conform to a shape of the groove.

The one or more capacitors may have any suitable maximum voltage to provide therapeutic electrical pulses. Each of the one or more capacitors may have an individual maximum voltage that can be the same or different from the individual maximum voltage of other capacitors of the one or more capacitors. At least one of the one or more capacitors may have a maximum voltage of, for example, at least 100 volts, at least 300 volts, at least 1000 volts, at least 2000 volts, or any voltage therebetween. In one or more embodiments, at least one capacitor of the one or more capacitors may have a maximum voltage of at least 1000 volts.

The one or more capacitors may have any suitable maximum capacitance to provide therapeutic electrical pulses. Each of the one or more capacitors may have an individual maximum capacitance that can be the same or different from the individual maximum capacitance of other capacitors of the one or more capacitors. At least one of the one or more capacitors may have a maximum capacitance of, for example, at least 15 Farads, at least 40 Farads, at least 300 Farads, or any capacitance therebetween. In one or more embodiments, at least one capacitor of the one or more capacitors may have a maximum capacitance of at least 300 Farads.

The use of the crystalline dielectric 126 in the one or more capacitors 116 may also allow the capacitors to have a greater energy density than typical capacitors used in pulse generators (e.g., electrolytic capacitors). The one or more capacitors 116 may have a higher maximum voltage than electrolytic capacitors with a volume less than or equal to the electrolytic capacitors. Accordingly, the one or more capacitors 116 may have a greater energy density because the energy density of capacitors is directly proportional to the square of the maximum voltage of the capacitors. At least one of the one or more capacitors may have an energy density of at least 10 joules per cubic centimeter, at least 30 joules per cubic centimeter, at least 50 joules per cubic centimeter, at least 70 joules per cubic centimeter, or at least 100 joules per cubic centimeter.

The pulse generator may be configured to deliver the electrical pulse with any suitable voltage. The pulse generator may be configured to deliver the electrical pulse with a voltage of, for example, at least 100 volts, at least 300 volts, at least 1000 volts, at least 2000 volts, or any voltage therebetween. In one or more embodiments, the pulse generator may be configured to deliver the electrical pulse with a voltage of at least 1000 volts. The pulse generator may include a step-up converter to boost or increase the voltage received from the power source. The step-up converter may be configured to increase the voltage received from the power source to a therapeutic pulse voltage. An output voltage of the step-up converter may be adjustable based on a desired therapeutic pulse voltage. For example, the controller may be configured to adjust the output voltage of the step-up converter based on the desired therapeutic pulse voltage.

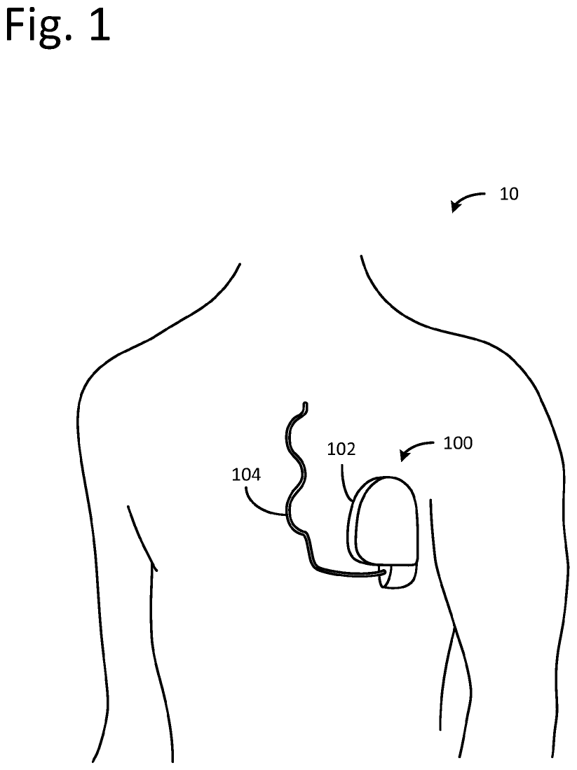

An embodiment of a therapeutic electrical pulse delivery system that includes crystalline capacitors as described herein, is depicted in FIG. 1. FIG. 1 shows a conceptual drawing illustrating the therapeutic electrical pulse delivery system 100 in conjunction with a patient 10. As depicted in FIG. 1, the therapeutic electrical pulse delivery system 100 includes a housing 102 that defines the exterior of an implantable medical device. The therapeutic electrical pulse delivery system 100 may be or may be included in, any suitable implantable medical device such as, for example, implantable pulse generators, implantable cardioverter defibrillators, implantable cardiac contractility modulators, implantable neurostimulators, implantable mechanical assist devices, etc.

The therapeutic electrical pulse delivery system 100 may also include one or more leads 104 to deliver therapeutic electrical pulses to desired treatment areas of the patient 10. The leads 104 may include one or more electrodes (not shown) to facilitate delivery of the therapeutic electrical pulses to the desired treatment areas. In one or more embodiments, the therapeutic electrical pulse delivery system 100 may include one or more electrodes without any leads. For example, when the therapeutic electrical pulse delivery system 100 can be implanted at the desired treatment area, leads may not be needed to deliver the therapeutic electrical pulses.

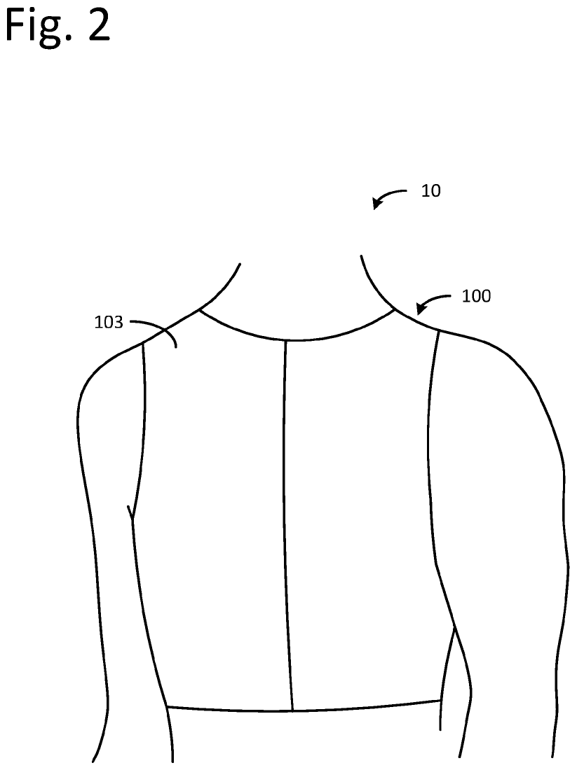

In addition to implantable medical devices, the therapeutic electrical pulse delivery system 100 may be, or may be housed in, wearable or other non-implantable devices. An example of the therapeutic electrical pulse delivery system 100 housed in a vest 103 is shown in FIG. 2. Other wearable devices may include cuffs, skin patches, etc. Wearable therapeutic electrical pulse delivery systems that use capacitors that include a crystalline dielectric may have reduced bulk and weight compared to therapeutic electrical pulse delivery systems that use other types of capacitors such as electrolytic capacitors.

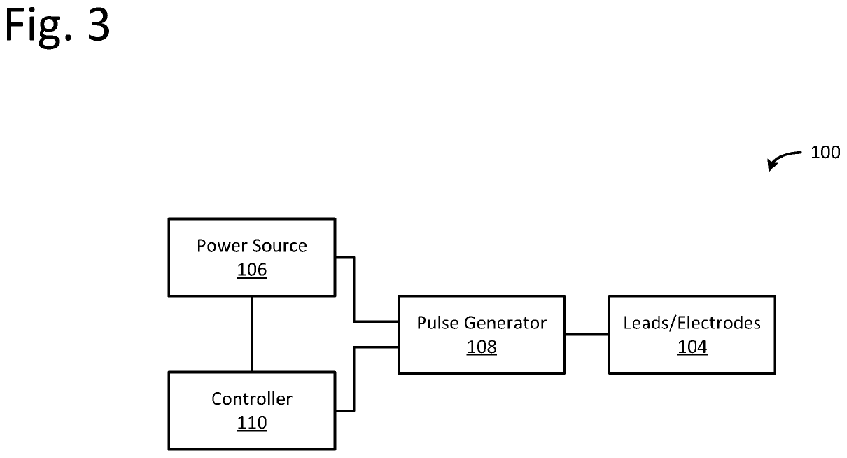

A schematic block diagram of the electronic components of the therapeutic electrical pulse delivery system 100 are depicted in FIG. 3. The electronic components of the therapeutic electrical pulse delivery system 100 can be disposed in the housing 102 to be implanted in the patient 10 as shown in FIG. 1 or disposed in a wearable device such as the vest 103 of FIG. 2. The therapeutic electrical pulse delivery system 100 may include a power source 106, a pulse generator 108, and a controller 110.

The power source 106 may be operatively coupled to the pulse generator 108 to provide energy to charge the pulse generator 108. In general, the power source 106 may be a voltage source. The power source 106 may be configured to provide energy at a voltage of greater than zero volts to about five volts. In some examples, the power source 106 is configured to provide energy at a voltage of about two volts to about four volts (e.g., about three volts). Accordingly, the current provided by the power source 106 to the pulse generator 108 may vary based on a state of charge of the pulse generator. The power source 106 may include any suitable energy storage and/or power delivery apparatus. The power source 106 may include one or more, for example, batteries, electrochemical cells, fuel cells, super capacitors, switches, controllers, battery management systems, or other energy storage and/or power delivery apparatus.

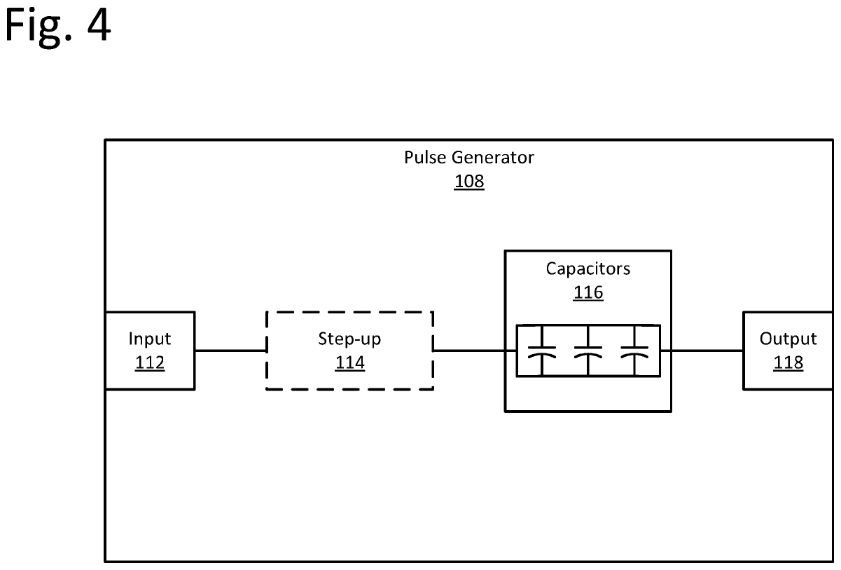

The pulse generator 108 may be operatively coupled to the power source 106. The pulse generator 108 may be configured to receive energy from the power source 106 within a nominal voltage range of the power source 106. A schematic block diagram of an embodiment of the pulse generator or therapeutic pulse generator 108 is depicted in FIG. 4. The pulse generator 108 may be operatively coupled to or operatively couplable to the power source 106 via an input 112. The input 112 may include a switch to allow control of the receipt of energy from the power source 106. Alternatively, the input 112 may be operatively coupled to an external switch. The external switch may be part of the pulse generator 108 generally or included in the power source 106. The switch, whether internal or external to the pulse generator, may include any suitable device or devices. The switch may include, for example, one or more transistors, electromechanical switches, toggles, etc.

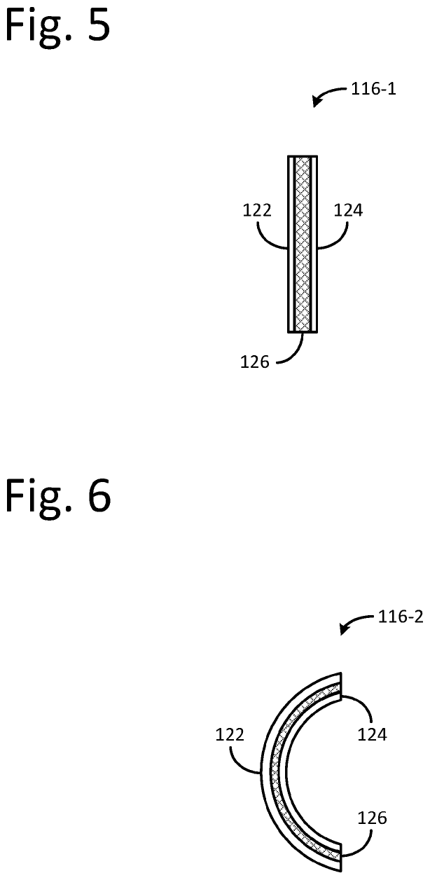

The pulse generator 108 may include one or more capacitors 116. Each of the one or more capacitors 116 may include a first electrode 122, a second electrode 124, and a crystalline dielectric 126 disposed between the first electrode 122 and the second electrode 124 as shown by capacitors 116-1, 116-2, 116-3, 116-4 (referred to collectively as the capacitors 116) in FIGS. 5-8. The crystalline dielectric 126 may include carbon. In other words, each of the one or more capacitors 116 may be a crystalline capacitor.

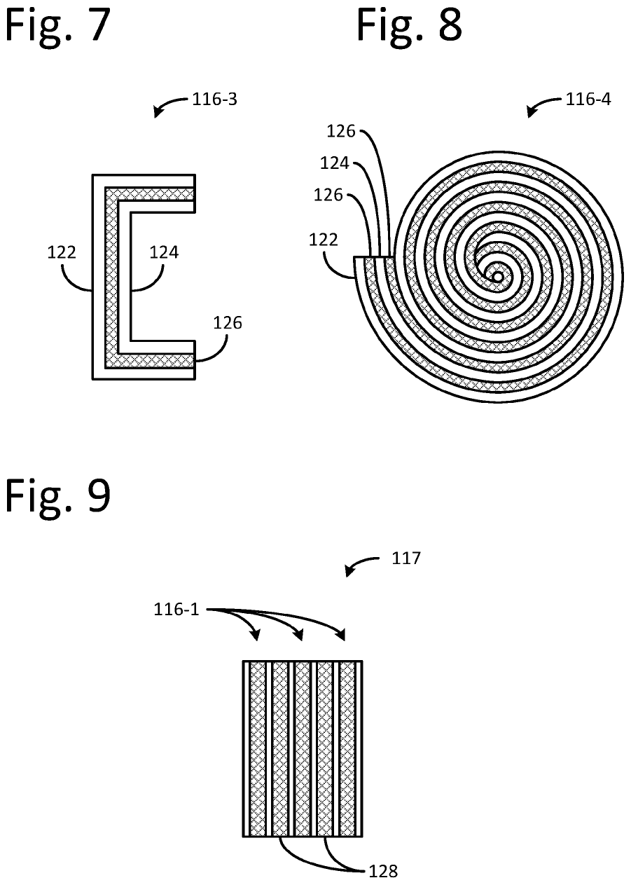

The use of a crystalline dielectric may allow the capacitors 116 to take on a variety of shapes and designs. The capacitors 116 may take on a variety of shapes and designs because the crystalline dielectric can be disposed on one or both of the electrodes 122, 124 in layers. In some configurations, the crystalline dielectric is deposited on one or both of the electrodes 122, 124 in layers. Some examples of the shapes the capacitors 116 can take on are depicted by the capacitors 116-1, 116-2, 116-3, 116-4 of FIGS. 5-8. In one example, the electrodes 122, 124 can be generally flat with the crystalline dielectric 126 disposed between them as shown by capacitor 116-1 of FIG. 5. Capacitors such as capacitor 116-1 may be formed by disposing alternating layers of metal and crystalline materials (e.g., diamond, silicon carbide, etc.) to form the electrodes 122, 124 and the crystalline dielectric. In another example, the electrodes 122, 124 can be curved with the crystalline dielectric 126 disposed between them as shown by capacitor 116-2 of FIG. 6. In yet another example, the electrodes 122, 124 can be bent at one or more angles with the crystalline dielectric 126 disposed between them as shown by capacitor 116-3 of FIG. 7. In other words, the capacitors 116 can conform to the shape of various housings or substrates. In still another example, the electrodes 122, 124 can be wound about an axis with the crystalline dielectric 126 disposed between them as shown by capacitor 116-4 of FIG. 8. Capacitors such as capacitor 116-4 may be formed by disposing crystalline materials on metal foils or sheets and winding the metal foils to form one or more windings defined by the first electrode 122, the second electrode 124, and the crystalline dielectric 126.

Additionally, the capacitors 116 may include a plurality of capacitors in a stacked arrangement 117 as shown in FIG. 9. The stacked arrangement 117 may include a plurality of the capacitors 116-1 (see FIG. 5) with additional crystalline dielectric 128 disposed between each of the plurality of the capacitors 116-1. Although shown with the capacitors 116-1, the stacked arrangement 117 can be formed using capacitors of any suitable shape (e.g., capacitors 116-2, 116-3, 116-4 of FIGS. 6-8) with additional crystalline dielectric 128 disposed therebetween.

The pulse generator 108 may include an output 118 operatively coupled to the one or more capacitors to deliver a therapeutic electrical pulse using energy stored in the one or more capacitors. The output 118 may include a switch to allow control delivery of the therapeutic electrical pulse from the one or more capacitors 116. The switch may include any suitable device or devices to control the delivery of the therapeutic electrical pulse. The switch may include, for example, one or more transistors, electromechanical switches, toggles, etc.

The pulse generator 108 may also include a step-up converter 114. The step-up converter 114 may be configured to increase the voltage of energy received from the power source 106. In one or more embodiments, the output voltage of the power source 106 may be lower than a desired voltage for delivery of a therapeutic electrical pulse. In such embodiments, the step-up converter 114 may boost the voltage provided by the power source 106. In one or more embodiments, the step-up converter 114 may be adjustable to allow the pulse generator 108 to deliver therapeutic electrical pulses at various voltages. For example, the step-up converter 114 may be configured to adjust an output voltage based on commands or signals received from the controller 110. The step-up converter 114 may include any suitable device or devices to boost or increase the voltage provided by the power source 106. The step-up converter 114 may include one or more of, for example, a direct current (DC)-DC converter, switches, transistors, transformers, inductors, etc.

Energy transfer from the power source 106 to the pulse generator 108 may be controlled by a controller 110. The controller 110 may be operatively coupled to the power source 106 and/or the pulse generator 108. The controller 110 may be configured to charge one or more capacitors 116 of the pulse generator 108 using the power source 106 and cause the pulse generator 108 to deliver a therapeutic electrical pulse using the charged one or more capacitors 116. To charge the one or more capacitors 116, the controller 110 may be configured to close a switch associated with the input 112 and/or the power source 106 to allow current to flow from the power source 106 to the pulse generator 108. To deliver the therapeutic electrical pulse, the controller 110 may be configured to close a switch associated with the output 118 of the pulse generator 108. Additionally, the controller 110 may be configured to open the switch of the input 112 or the power source 106 before closing the switch of the output 118. When the switch of the output 118 is closed, energy stored in the capacitors 116 may flow through the output to one or more leads/electrodes 104 and ultimately to therapy delivery sites of the patient 10.

The controller 110 may include any suitable analogue or digital circuitry to charge the one or more capacitors 116 and deliver therapeutic electrical pulses. The controller may include, for example, one or more processors, logic gates, operational amplifiers, transistors, analogue-to-digital converters, sensors, or other circuitry or devices to control the power source 106 and/or the pulse generator 108. The controller 110 may include data storage for data storage and access to processing programs or routines that may be employed to carry out the techniques, processes, and algorithms for charging the one or more capacitors 116 and delivering a therapeutic electrical pulse. For example, processing programs or routines may include programs or routines for pulse delivery timing, pulse delivery triggers, opening and closing switches, determining an output voltage, adjusting an output voltage, filtering background noise, computational mathematics, matrix mathematics, Fourier transforms, compression algorithms, calibration algorithms, inversion algorithms, signal processing algorithms, normalizing algorithms, deconvolution algorithms, averaging algorithms, standardization algorithms, comparison algorithms, vector mathematics, or any other processing required to implement one or more embodiments as described herein.

The controller 110 may also include a communication interface to communicate with one or more external devices. The communication interface may include any suitable hardware or devices to provide wired or wireless communication with the one or more external devices. For example, the communication interface may include one or more of a receiver, transmitter, transceiver, ethernet port, Universal Serial Bus (USB) port, cables, controller, or other device to facilitate wired or wireless communication. The communication interface may facilitate communication using any suitable protocol or protocols. For example, the communication interface may utilize Ethernet, Recommended Standard 232, Universal Asynchronous Receiver Transmitter or Universal Synchronous Asynchronous Receiver Transmitter (UART/USART), USB, BLUETOOTH, Wi-Fi, Near Field Communication (NCF), etc. The communication interface may allow communication between the therapeutic electrical pulse delivery system 100 and a computing apparatus.

The invention is defined in the claims. However, below there is provided a non-exhaustive list of non-limiting examples. Any one or more of the features of these examples may be combined with any one or more features of another example, embodiment, or aspect described herein.

Example Ex1: A therapeutic electrical pulse delivery system comprising: a power source; a pulse generator operatively coupled to the power source, the pulse generator comprising one or more capacitors, each of the one or more capacitors comprising: a first electrode; a second electrode; and a crystalline dielectric disposed between the first electrode and the second electrode, the crystalline dielectric comprising carbon; and a controller comprising one or more processors and operatively coupled to the power source or the pulse generator, the controller configured to: charge the one or more capacitors of the pulse generator using the power source; and cause the pulse generator to deliver a therapeutic electrical pulse using the charged one or more capacitors.

Example Ex2: The therapeutic electrical pulse delivery system as in example Ex1, wherein the pulse generator further comprises: an input operatively coupled to the power source and configured to charge the one or more capacitors in parallel; and an output operatively coupled to the one or more capacitors and configured to selectively deliver the therapeutic electrical pulse from the one or more capacitors in series, parallel, or in a combination of series and parallel.

Example Ex3: The therapeutic electrical pulse delivery system as in example Ex1, wherein the one or more capacitors consists of a single capacitor.

Example Ex4: The therapeutic electrical pulse delivery system as in any one of examples 1 or 2, wherein the one or more capacitors comprises a plurality of capacitors in a stacked arrangement.

Example Ex5: The therapeutic electrical pulse delivery system as in any one of the previous examples, wherein the first electrode, the second electrode, and the crystalline dielectric of at least one of the one or more capacitors define a curved shape.

Example Ex6: The therapeutic electrical pulse delivery system as in any one of the previous examples, further comprising a substrate comprising a groove in at least one surface of the substrate, wherein the one or more capacitors conform to a shape of the groove.

Example Ex7: The therapeutic electrical pulse delivery system as in any one of the previous examples, wherein at least one capacitor of the one or more capacitors has a maximum voltage of at least 1000 volts.

Example Ex8: The therapeutic electrical pulse delivery system as in any one of the previous examples, wherein the pulse generator is configured to deliver the electrical pulse with a voltage of at least 1000 volts.

Example Ex9: The therapeutic electrical pulse delivery system as in any one of the previous examples, wherein the crystalline dielectric comprises diamond.

Example Ex10: The therapeutic electrical pulse delivery system as in any one of the previous examples, wherein at least one capacitor of the one or more capacitors has an energy density of at least 10 joules per cubic centimeter.

Example Ex11: The therapeutic electrical pulse delivery system as in any one of the previous examples, further comprising a housing, wherein the pulse generator is disposed in the housing.

Example Ex12: The therapeutic electrical pulse delivery system as in example Ex11, wherein the housing comprises a vest.

Example Ex13: An implantable medical device comprising the therapeutic electrical pulse delivery system as in any one of examples Ex1 to Ex11.

Example Ex14: A therapeutic pulse generator comprising: an input operatively couplable to a power source; one or more capacitors coupled to the input to receive and store energy provided by the power source, each of the one or more capacitors comprising: a first electrode; a second electrode; and a crystalline dielectric disposed between the first electrode and the second electrode, the crystalline dielectric comprising carbon; and an output operatively coupled to the one or more capacitors to deliver a therapeutic electrical pulse using energy stored in the one or more capacitors.

Example Ex15: The therapeutic pulse generator as in example Ex14, wherein the input is configured to charge the one or more capacitors in parallel and the output is configured to selectively deliver the therapeutic electrical pulse from the one or more capacitors in series, parallel, or in a combination of series and parallel.

Example Ex16: The therapeutic pulse generator as in example Ex14, wherein the one or more capacitors consists of a single capacitor.

Example Ex17: The therapeutic pulse generator as in any one of examples Ex14 or Ex15, wherein the one or more capacitors comprises a plurality of capacitors in a stacked arrangement.

Example Ex18: The therapeutic pulse generator as in any one of examples Ex14 to Ex17, wherein the first electrode, the second electrode, and the crystalline dielectric of at least one of the one or more capacitors define a curved shape.

Example Ex19: The therapeutic pulse generator as in any one of examples Ex14 to Ex18, further comprising a substrate comprising a groove in at least one surface of the substrate, wherein the one or more capacitors conform to a shape of the groove.

Example Ex20: The therapeutic pulse generator as in any one of examples Ex14 to Ex19, wherein at least one capacitor of the one or more capacitors has a maximum voltage of at least 1000 volts.

Example Ex21: The therapeutic pulse generator as in any one of examples Ex14 to Ex20, wherein the pulse generator is configured to deliver the electrical pulse with a voltage of at least 1000 volts.

Example Ex22: The therapeutic pulse generator as in any one of examples Ex14 to Ex21, wherein the crystalline dielectric comprises diamond.

Example Ex23: The therapeutic pulse generator as in any one of examples Ex14 to Ex22, wherein at least one capacitor of the one or more capacitors has an energy density of at least 10 joules per cubic centimeter.

Example Ex24: The therapeutic pulse generator as in any one of examples Ex14 to Ex23, further comprising a step-up converter operatively coupled to the input and the one or more capacitors to step-up a voltage received from the power source. All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A therapeutic electrical pulse delivery system comprising:
a power source;
a pulse generator operatively coupled to the power source, the pulse generator comprising one or more capacitors, each of the one or more capacitors comprising:
a first electrode;
a second electrode; and
a crystalline dielectric disposed between the first electrode and the second electrode, the crystalline dielectric comprising carbon; and
a controller comprising one or more processors and operatively coupled to the power source or the pulse generator, the controller configured to:
charge the one or more capacitors of the pulse generator using the power source; and
cause the pulse generator to deliver a therapeutic electrical pulse using the charged one or more capacitors.

2. The therapeutic electrical pulse delivery system as in claim 1, wherein the pulse generator further comprises:
an input operatively coupled to the power source and configured to charge the one or more capacitors in parallel; and
an output operatively coupled to the one or more capacitors and configured to selectively deliver the therapeutic electrical pulse from the one or more capacitors in series, parallel, or in a combination of series and parallel.

3. The therapeutic electrical pulse delivery system as in claim 1, wherein the one or more capacitors consists of a single capacitor.

4. The therapeutic electrical pulse delivery system as in claim 1, wherein the one or more capacitors comprises a plurality of capacitors in a stacked arrangement.

5. The therapeutic electrical pulse delivery system as in claim 1, wherein the first electrode, the second electrode, and the crystalline dielectric of at least one of the one or more capacitors define a curved shape.

6. The therapeutic electrical pulse delivery system as in claim 1, further comprising a substrate comprising a groove in at least one surface of the substrate, wherein the one or more capacitors conform to a shape of the groove.

7. The therapeutic electrical pulse delivery system as in claim 1, wherein at least one capacitor of the one or more capacitors has a maximum voltage of at least 1000 volts.

8. The therapeutic electrical pulse delivery system as in claim 1, wherein the pulse generator is configured to deliver the electrical pulse with a voltage of at least 1000 volts.

9. The therapeutic electrical pulse delivery system as in claim 1, wherein the crystalline dielectric comprises diamond.

10. The therapeutic electrical pulse delivery system as in claim 1, wherein at least one capacitor of the one or more capacitors has an energy density of at least 10 joules per cubic centimeter.

11. The therapeutic electrical pulse delivery system as in claim 1, further comprising a housing, wherein the pulse generator is disposed in the housing.

12. The therapeutic electrical pulse delivery system as in claim 11, wherein the housing comprises a vest.

13. An implantable medical device comprising the therapeutic electrical pulse delivery system as in claim 1.

14. A therapeutic pulse generator comprising:
an input operatively couplable to a power source;
one or more capacitors coupled to the input to receive and store energy provided by the power source, each of the one or more capacitors comprising:
a first electrode;
a second electrode; and
a crystalline dielectric disposed between the first electrode and the second electrode, the crystalline dielectric comprising carbon; and
an output operatively coupled to the one or more capacitors to deliver a therapeutic electrical pulse using energy stored in the one or more capacitors.

15. The therapeutic pulse generator as in claim 14, wherein the input is configured to charge the one or more capacitors in parallel and the output is configured to selectively deliver the therapeutic electrical pulse from the one or more capacitors in series, parallel, or in a combination of series and parallel.

16. The therapeutic pulse generator as in claim 14, wherein the one or more capacitors consists of a single capacitor.

17. The therapeutic pulse generator as in claim 14, wherein the one or more capacitors comprises a plurality of capacitors in a stacked arrangement.

18. The therapeutic pulse generator as in claim 14, wherein the first electrode, the second electrode, and the crystalline dielectric of at least one of the one or more capacitors define a curved shape.

19. The therapeutic pulse generator as in claim 14, further comprising a substrate comprising a groove in at least one surface of the substrate, wherein the one or more capacitors conform to a shape of the groove.

20. The therapeutic pulse generator as in claim 14, wherein at least one capacitor of the one or more capacitors has a maximum voltage of at least 1000 volts.

21. The therapeutic pulse generator as in claim 14, wherein the pulse generator is configured to deliver the electrical pulse with a voltage of at least 1000 volts.

22. The therapeutic pulse generator as in claim 14, wherein the crystalline dielectric comprises diamond.

23. The therapeutic pulse generator as in claim 14, wherein at least one capacitor of the one or more capacitors has an energy density of at least 10 joules per cubic centimeter.

24. The therapeutic pulse generator as in claim 14, further comprising a step-up converter operatively coupled to the input and the one or more capacitors to step-up a voltage received from the power source.
